(19) United States
(12) Patent Application Publication
(10) Pub. No.: US 2009/0326611 A1
(43) Pub. Date: Dec. 31, 2009

(54) Method of Powering Implanted Devices by Direct Transfer of Electrical Energy

(76) Inventor: Ivor Stephen Gillbe, Bristol (GB)

Correspondence Address: MCGARRY BAIR PC, 32 Market Ave. SW, SUITE 500, GRAND RAPIDS, MI, 49503, US

(21) Appl. No.: 12/374,901

(22) PCT Filed: Jul. 25, 2007
(86) PCT No.: PCT/GB07/02814

§ 371 Date: Jan. 23, 2009

(30) Foreign Application Priority Data
Jul. 25, 2006 (GB) 0614777.1

(51) Int. Cl. A61N 1/378

(52) U.S. Cl. 607/61

(57) ABSTRACT

In order to transfer electrical energy to an implemented medical device (04) power electrodes (02) are fitted in contact with the body of a human or animal into which the medical device has been implanted. The power electrodes (02) may be directly on the skin of the body or may penetrate the skin. The implanted medical device (04) has implanted electrodes (03) which receive electrical energy via the body. To power the implanted medical device (04), a power device (01) applies an electric potential in the form of a repetitive waveform to the power electrodes (02), thereby generating an electric current in the body, and transferring electrical energy via the implanted electrodes (03) to the implanted medical device (04). Preferably the waveform is a pulsed waveform with pulses of a duration not greater than 8 μs and an inter-pulse spacing not greater than 20 μs.

### BACKGROUND OF THE INVENTION

1. Field of the Invention

The present invention relates to implantable medical devices and in particular to energy transfer to these devices by passing an electric current through the tissues.

2. Summary of the Prior Art

Implantable medical devices for producing a therapeutic result are well known. Examples include cardiac pacemakers, infusion pumps, neurostimulators, cochlear implants and implanted monitoring devices. All such devices require a power source and in many cases this is provided by electrical power either from an internal battery or from an external source.

Known methods of coupling external power to an implanted device include magnetic induction wherein an externally generated magnetic field is coupled to an internal inductor as exemplified by Olson et al U.S. Pat. No. 2005/0075699 “System and method for transcutaneous energy transfer achieving high efficiency” (Medtronic Inc) and references therein. This patent discloses an external power source having a primary coil and an implanted secondary coil. By careful positional adjustment of the primary and secondary coils the inventors claim an efficiency of energy transfer of at least 30%.

Meadows et al European Patent No EP1518584 “Rechargeable stimulator system” (Advanced Bionics Corporation) discloses a method of charging a rechargeable battery carried within an implantable pulse generator (IPG), the IPG having a secondary coil antenna through which electromagnetic energy may be coupled to non-invasively transfer energy into the IPG.

Other methods of inductive charging have been envisaged where the external charging coils are not placed directly over the skin, such as Carbunaru et al US Patent 2004/098068 “Chair pad charging and communication system for a battery-powered microstimulator” (Advanced Bionics Corporation) and Schulman et al WO03039652 “Full body charger for battery powered patient implantable device” which discloses a bed with a plurality of transmitting coils such that the patient's device might be recharged during sleep. While apparently convenient, these systems suffer from very low efficiency when compared to systems where the receiving and transmitting coils are in close proximity.

There are many other examples of implantable systems charged by inductive devices and improvements thereon.

A practical disadvantage of systems with devices that contain induction coils is that the implanted induction coil may heat-up during charging. Any increase in tissue temperature around the implant beyond 2-3 C may be deleterious. Furthermore, in order to achieve reasonable levels of efficiency, the receiving induction coil should be as close to the skin as possible, typically at a recommended depth of 5-10 mm, which means that the device may be visible under the skin.

Another method of coupling magnetic energy to the device is described in Schroeppel and Spehrus U.S. Pat. No. 5,749,909 “Transcutaneous energy coupling using piezoelectric device” (Sulzer Intermedics Inc) which discloses an energy transmission system for transmitting energy non-invasively from an external unit to an implanted medical device to recharge a battery in the medical device. An alternating magnetic field is generated by the external charging unit and a piezoelectric device in the implanted medical device vibrates in response to the magnetic flux to generate a voltage.

An alternative technique for powering implanted medical devices is to extract waste energy from the environment, often referred to as “energy scavenging”. Examples include devices that use vibration to excite piezoelectric generators or thermopiles to extract energy from the temperature gradient in the tissues. Macdonald U.S. Pat. No. 6,640,137 “Biothermal power source for implantable devices” (Biomed Solutions LLC) describes a thermal device that generates 100 microwatts from a 1 degree C. temperature differential, sufficient to power a sensor device but not active therapeutic devices such as neurostimulators.

### Nerve Stimulation Background

Direct electrical stimulation of the tissues has been in common use for therapeutic purposes for the past 30 years. In 1965 Melzack and Wall 1965 described how analgesia could be produced when Aβ fibers are stimulated at 100 Hz, a frequency that none of the other sensory nerves can follow faithfully. Wall employed surface electrodes, leading to the term Transcutaneous Electrical Nerve Stimulation (TENS).

Woolf 1989 reviewed the use of these devices, and described their electrical parameters. The usual TENS machine develops a pulse, the width of which can be varied from 50 to 500 μs, employing a current of amplitude 0 to 50 mA, and frequency is generally 100 Hz.

As tissue impedance is capacitive, it tends to fall as frequency is increased. In order to increase tissue penetration, signals may be provided at a frequency where the intervals between each electric signal are less than the refractory periods of axons that require stimulation. In order to produce action potentials, such signals are modulated to provide low frequency stimulation either by interference or interruption.

The interference method of applying medium frequency currents is exemplified by Nemec U.S. Pat. No. 2,622,601 “Electrical Nerve Stimulator”, Griffith U.S. Pat. No. 3,096,768 “Electrotherapy System” (Firmtron Inc) and many others. Two signal sources are each connected to a pair of electrodes. They can produce an amplitude modulated medium frequency signal in the tissues called interferential current, as follows. The first signal source uses a medium frequency carrier wave (typically 4.0 kHz) while the other operates at a slightly different frequency (typically 4.1 kHz). Their respective pairs of surface electrodes are arranged on the body in a manner that allows the two oscillating currents to intersect in deep tissues where interference is produced at a beat frequency in the low frequency range, typically at 100 Hz. This in turn is said to stimulate deeply placed Aβ fibers to produce analgesia.

Macdonald and Coates U.S. Pat. No. 5,776,170 “Electrotherapeutic apparatus” explored the effects of applying electric signals whose pulse width is so brief, typically 4 μs, that the voltage gated channels lying in excitable membranes of peripheral nerve axons that lie in the path of the current do not have time to respond to these signals sufficiently to reach membrane threshold and produce action potentials.

Littlewood et al WO2005115536 “Electrotherapy Apparatus” (Bioinduction Ltd) discusses the effects of employing short high power electrotherapy waveforms for therapeutic purposes and describes the relationship between pulse width and the generation of action potentials and shows that the current to the tissues may controlled independently of the level of sensation felt by the patient.

### SUMMARY OF THE INVENTION

The present invention provides a system for transcutaneous energy transfer to an implanted medical device, using electrical energy applied to the skin from an external power unit. The received energy may be used either for charging an implanted battery or for providing energy directly to an implanted device.

In the present invention, at its most general, the electrical energy is transmitted as a repetitive waveform preferably, consisting of pulses of short duration, so short that the peripheral nerves cannot respond and consequently there is no sensation of current flowing.

The invention employs electrodes in direct electrical contact with the tissues of the body, either using electrodes that are applied directly to the skin, or electrodes that make contact by penetrating the skin. In the former case, the electrodes may generally consist of a conductive substrate, such as carbon rubber or conductive metallic layer, with a hydro-gel or other water based layer that provides an electrical interface with the skin. In the latter case, the electrodes may be provided with a number of miniature needles that penetrate the resistive outer layer of the skin thereby improving electrical contact with the tissues below. A final alternative is to provide electrodes in the form of longer needles that directly penetrate the skin, making electrical contact with the tissues below. The invention differs from known inductive methods of charging in which energy is transferred via a pulsed magnetic or electromagnetic waveform, in that an electrical current is applied directly to the tissues.

Thus, a first aspect of the invention may provide an implanted system comprising:

a power device external to a human or animal body, the power device having power electrodes in contact with the body so as to make direct electrical connection to the tissues of the body, the power device being arranged to apply electrical energy to the body via the power electrodes; and
an implanted device within the body, the implanted device having implanted electrodes arranged to receive said electrical energy via the body, thereby to provide power for said implanted device,
wherein the power device is arranged to apply an electric potential between the power electrodes, the potential being in the form of a repetitive waveform, the electric potential being such as to generate electric current in the body and thereby to transfer said electrical energy to said implanted electrodes.

Preferably the waveform is a pulsed waveform, e.g. with a zero amplitude at some point in its time cycle. The pulses can be of any shape, including square and sinusoidal, continuous or discontinuous, and alternating in polarity on all having the same polarity. However, other waveforms may be possible, for example by imposing a D.C. component or a sinusoidal or square waveform so that the minimum current is non-zero but sufficiently small that the peripheral nerves are unaffected.

Where the waveform is pulsed it may be delivered in a interrupted form consisting of high voltage pulses of typically 0.5 to 4 or 8 μs duration at an amplitude which may be up to 250 V or more. Each pulse or group of pulses may be delivered at a low duty cycle separated by quiet periods so that losses in the tissues do not cause appreciable heating. Furthermore, the short pulses should normally be short enough, preferably less than 8 us and more preferably less than 2 μs, so that the series capacitance of the tissues does not cause the applied voltage to decay significantly during the pulse.

The pulses can be of any shape but a square wave is preferred particularly for implanted devices that do not incorporate a transformer or other signal amplification means on the input. It is also preferred that the polarity of the pulsed waveform alternates between positive and negative pulses. Then the waveform should preferably have balancing positive and negative pulses of equal charge so that there is no net movement of ions from one electrode to the other, either on the surface or in the flesh. Preferably the balancing negative pulse should immediately follow the forward pulse because this increases the amplitude of the pulse current that can be applied without activating peripheral nerves by a factor of about three for the short pulses considered here. If there is an inter-pulse spacing, it should preferably be not greater than 20 μs, more preferably not greater than 10 μs and ideally 0 μs.

Preferably the implanted electrodes are positioned under the skin in the proximity of the surface electrodes because this allows maximum efficiency of energy transfer. The implanted electrodes may be constructed of a flexible material such as a thin conductor or wire mesh, so that they can be in close proximity the skin without being cosmetically obvious. One of the disadvantages of the existing induction loop rechargeable systems is that at their current recommended implantation depths, the outline of the implanted device may be visible under the skin.

If required, the implanted electrodes may also form part of the casing of the implant device, particularly if the device is 2 to 3 cm or less below the surface of the skin.

For low power implanted devices such as sensors, the implanted electrodes need not be directly under the skin. Electrical pulses provided by the external power unit will penetrate deep tissues. For example, it is straightforward to generate from surface electrodes 5 V or 10 V at a few milliwatts in the proximity of the heart or in the spinal canal.

If desired, the external power unit may be combined with the electrode pads to provide an integrated unit that is placed on the skin. The external electrode pads may be of self-adhesive type, or any other material that provides good electrical contact with the skin, such as conductive rubber pads with moistened sponge pads, or metal contacts in combination with a hydro-gel.

By selection of pulse width and repetition frequency, the external power unit may provide in the region of 0.2 W, after losses in the tissues, to an implanted device in a typical application, without significant heating of the tissues in the region of the implant. The transfer efficiency may be in the region of 10 to 20% in an optimized system.

An advantage of the technique is that the external power unit can directly deliver to the implant the voltage required to charge a battery or to power implanted electronic devices having no internal energy storage. Only simple circuitry in the implanted device is required which means that very compact implanted devices are possible using the invention. There is also minimal power dissipation in the implanted device itself, whereas known induction based devices suffer from heating of the induction coil and energy losses due to eddy currents in the device casing. The current invention employs receiving electrodes that may be more compact than induction coils, and may consist of a mesh or very fine biocompatible metal, such as titanium or platinum, designed so that they may be implanted under the skin without cosmetic issues.

As mentioned above, the preferred form of waveform for implanted devices of minimal complexity is the interrupted form because this has the advantage that the voltage at the implant may be higher. The voltage at the implant may then be such that a simple rectification and smoothing stage is all that is required to provide the charging current for a battery, or to provide the power to drive the device directly. The latter may be appropriate for implants that are normally passive but require power either for adjustment or for taking measurements. An example might be an implanted remotely adjustable gastric band, or a sensor which is powered up intermittently to take a reading.

A higher voltage at the implant has the advantage that a transformer or other step up circuit is not required in the implant and consequently energy losses in the implanted device are reduced. This means that the amount of energy that needs to be transmitted through the tissues is reduced. Small efficiency gains in the implant are important since the overall transfer efficiency through the tissues is relatively low. Therefore a small power loss in the implant is significant in terms of increased input power through the skin to make up the loss and the consequent heating effect of the transmission losses in the flesh. Clearly, the interrupted waveform requires a more sophisticated external power unit, but this additional complexity in the external device is outweighed by the simplicity and consequent reliability benefits, and by the smaller size of the implant.

Square waves are preferred in the example described above because with a simple receiving circuit in the implant it is possible to deliver energy at a constant voltage throughout the pulse. This simplifies the design of for instance battery charging circuits and reduces any losses for example in rectifying diodes in the implanted device.

If battery charging is required, it is preferable that the complexity in the charging circuit is external to the implant, in the device external to the body. This is possible with the invention disclosed herein by providing feedback from the implant about the received voltage, battery state and the rate of current flow. The external power unit can then determine the correct waveform duty cycle and voltage to deliver the desired voltage to the implant while maximizing the power delivered within the limits of heating of the tissues. Thus, the implanted device may include means for detecting the electrical power received by the implanted electrodes, means for transmitting to the power device information relating to said received power, and the power device has means for controlling the electrical energy applied to the body in response to said information.

As most heating occurs in the immediate proximity of the skin, the external power unit may also have temperature sensors built into the device, the electrode pads or a strap that is used to secure the device and electrodes in place. The sensor or sensors are used to measure the temperate rise in the tissues and modify the power input accordingly. Thus, the system may include a temperature sensor for detecting the temperature of the body adjacent the power electrodes, and means for controlling the electric energy applied to the body in dependence on said temperature.

In another aspect of the invention, very simple implanted neurostimulation devices are possible that include only passive components such as an isolation, rectification and smoothing stage and one or two stimulating electrodes. For example, the external power unit may generate bursts of high frequency energy which are received via implanted electrodes and delivered to stimulation electrodes after rectification and smoothing. The burst of high frequency energy would not cause the nerves under the surface electrodes to be stimulated, whereas after rectification and smoothing the resultant pulse signal would be of duration and amplitude to cause the nerves to be activated in the region of the implanted electrodes. In the case of such devices where the electrical connections to the tissues include implanted receiving electrodes and implanted stimulating electrodes, an isolating transformer on either the input or the output is desirable to prevent cross-coupling between the stimulating and receiving electrodes. The implant may optionally include a microprocessor device to measure and feed back information about the output signal to the external power unit. The size of such an implant would be such that the rectification and smoothing stage may be combined with the electrodes, which may be implanted in for instance the upper arm for a deep brain stimulation application, with the external power and control unit affixed to an arm-band.

Data exchange to and from the implanted device may be provided via radio telemetry or by encoding a signal in the waveform that is supplying the power or in the impedance seen by the device. In the case of transmission from an external power unit to the implant, the signal may be encoded in some form of modulation, for example pulse width, pulse code, amplitude or frequency modulation either applied directly to the waveform or using a higher frequency carrier that is mixed with the waveform. For transmission from the implant to the device, the low efficiency energy transfer through the tissues and the limited energy storage capability of the implant means that it is preferable that the implant does not directly drive current into the tissues. Instead, a more efficient means of communication from implant to external device is to vary the input impedance of the implant with a signal that contains encoded data, which can be sensed by circuitry in the external power unit.

Of the techniques that encode data in the applied electrical signal, pulse code modulation is preferred since it is easy to detect at the implant and also easy for the external power unit to detect impedance variations on the input to the implant used as described to send information back from the implant. Nevertheless, a more preferred means of communication is to use low power radio compatible with the Medical Implant Communications Service (MICS, 402-405 MHz) band as this uses readily available technology.

In order to maximize the efficiency of transfer, it is desirable that the surface electrodes are positioned over or near the implanted electrodes, particularly where the implanted electrodes are implanted within a few centimeters of the skin.

In this case, it may be desirable to have a search device that may be built into the external power unit that is able to direct the user to place the surface electrodes in the most advantageous position. A way of doing this is to have a detector system that transmits trial pulses into the skin from an array of electrodes. The array is constructed so that by comparing the voltage at the implant from different combinations of two or more surface electrodes in the array, it is possible to determine the direction and orientation of the implant relative to the surface electrode. The voltage at the implant is determined by sensing electronics in the implant and this information is transmitted wirelessly to the external device. Alternately, the external device can operate without communication with the implanted device by measuring changes in impedance of the combination of tissues and device at the array.

In a typical embodiment, the search device would have a display that shows the direction and orientation of the implant and directs the user to move the search device over the skin until the search device is centered over the implanted electrodes. In the search technique described, the center refers to the electrical centre rather than the physical centre. It will be appreciated that differences in tissue impedance may be such that the physical center position between two electrodes may be different from the position on the skin that provides the lowest impedance path to the electrodes. Clearly however, the latter is the desired positioning of the surface electrodes for power transmission efficiency. To make it easy to move the device while the search is in progress, metallic contacts on the search instrument rather than self adhesive contacts are preferred. A gel may be used to improve contact between electrode and skin.

An alternate method of finding advantageous positioning for the external electrodes is to use an array of multiple electrodes that are placed over the general area of the implant, or an array of metal or rubberized contacts on a belt or garment. Before commencing supplying power or during the supply of power, the external power unit would test the efficiency of transfer between various combinations of electrodes and the implanted device, in order to find the optimum configuration.

A more sophisticated approach of automatically optimizing energy transfer from an array of surface electrodes to a pair of implanted electrodes would be to apply signal processing techniques to derive drive coefficients for each electrode, the coefficients being amplitude and possibly phase (or pulse delay from a reference point). The process is similar to that used to optimize an adaptive equalizer as used for example in telecommunications receiving devices such as modems. In practice features of the received signal (including the amplitude and possibly including the phase and/or the waveform) are measured in the implanted device and conveyed by some means, telemetry for example, to the external device. The external device varies the amplitudes and delays of the pulses supplied to each element of an electrode array while keeping its total delivered output power (as measured at the electrode array) within appropriate limits. By matrix inversion or other mathematical or signal processing technique the coefficients for each element may be determined and set to maximize the ratio of received to transmitted power, thus optimizing not only for the effects of conduction in the skin and flesh but also (within limits) for the spatial adjustment of the electrodes and for any time-dependent effects. Since most of the additional circuitry may be contained in the external device the additional burden on the implanted device is minimized.

According to another aspect of the invention there is provided a method of providing power to an implanted device implanted within a human or animal body, the implanted device having implanted electrodes; the method comprising

locating power electrodes of a power device in contact with the body so as to enable direct electrical connection to tissues of the body, the power device being external to the body;
applying electrical energy to the body via the power electrodes by applying an electric potential between the power electrode, the electric potential being in the form of a repetitive waveform, thereby generating electric currents in the body;
receiving said electric current at said implanted electrodes, thereby conveys said electric energy to said electrodes, thereby to power said implanted device.

Specific embodiments of certain aspects of the present invention will now be described in more detail with reference to the figures. These are provided by way of explanation and example, and are not to be construed as limiting.

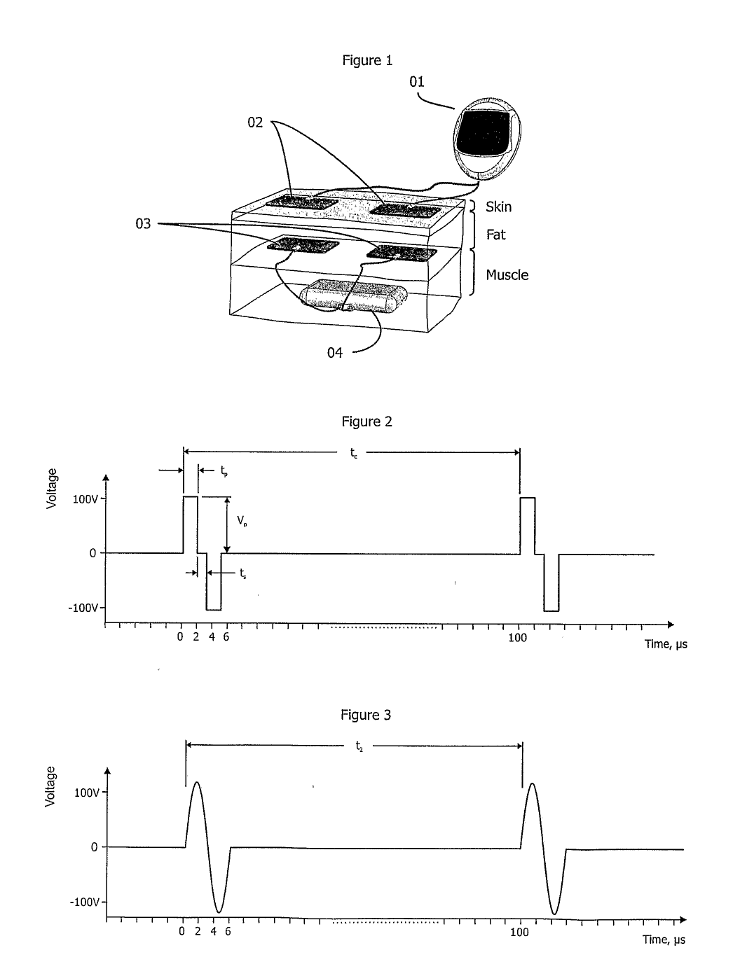

FIG. 1 illustrates a typical configuration of external and internal electrodes according to the invention with expanded scale in the direction perpendicular to the skin.

FIG. 2 illustrates a typical single cycle biphasic square waveform, of low duty cycle, having multiple biphasic cycles separated by quiet periods. This shows a waveform of amplitude just over +/−100 V but it should be noted that the peak may be +/−250 V or more.

FIG. 3 illustrates a typical single cycle sine wave, of low duty cycle.

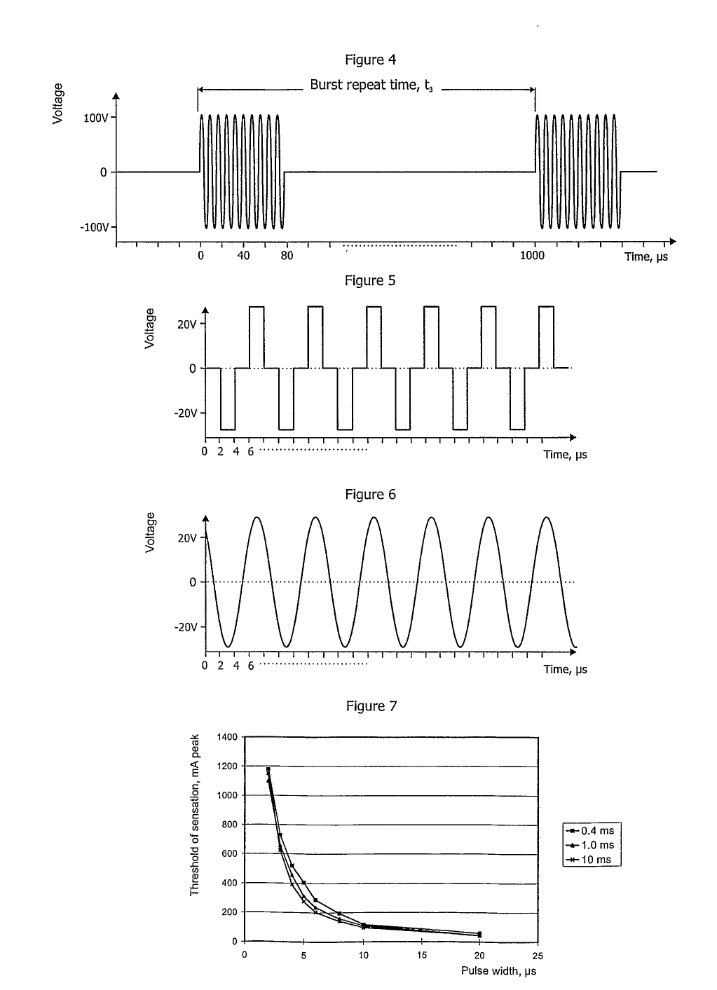

FIG. 4 illustrates a burst waveform.

FIG. 5 illustrates a typical continuous square waveform.

FIG. 6 illustrates a typical continuous sinusoidal waveform.

FIG. 7 illustrates the relationship between onset of sensation and peak pulse current at different pulse widths and three cycle repeat periods with zero inter-pulse spacing.

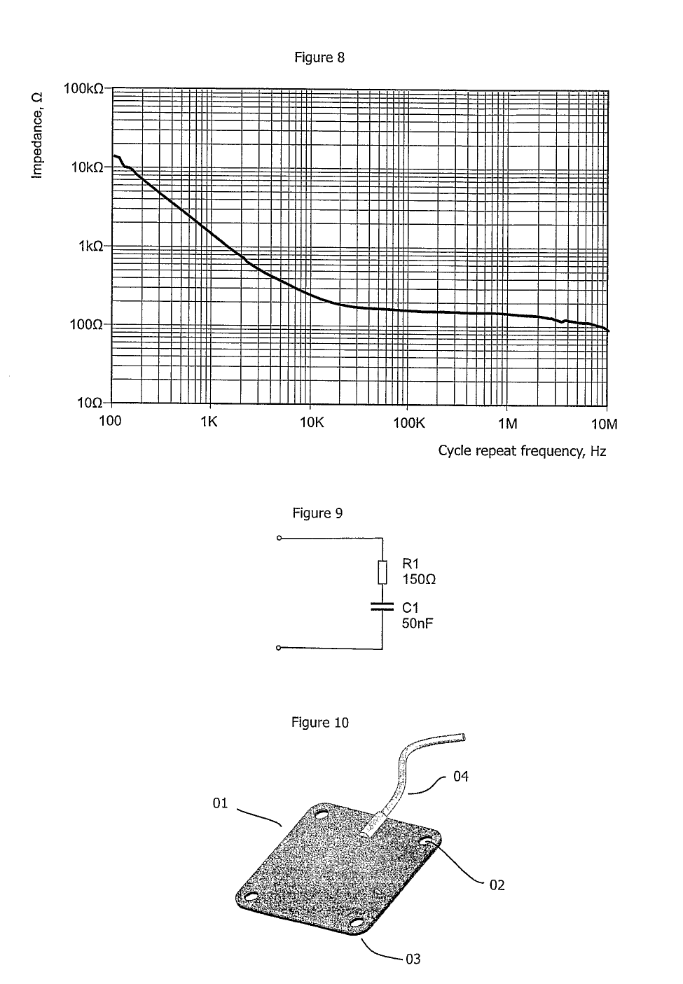

FIG. 8 shows a body impedance measurement using a sine wave input.

FIG. 9 shows a body equivalent circuit neglecting lead and electrode capacitance.

FIG. 10 illustrates an implanted receiving electrode with insulating undersurface and suture holes with the scale expanded in the direction normal to the conducting surface for clarity.

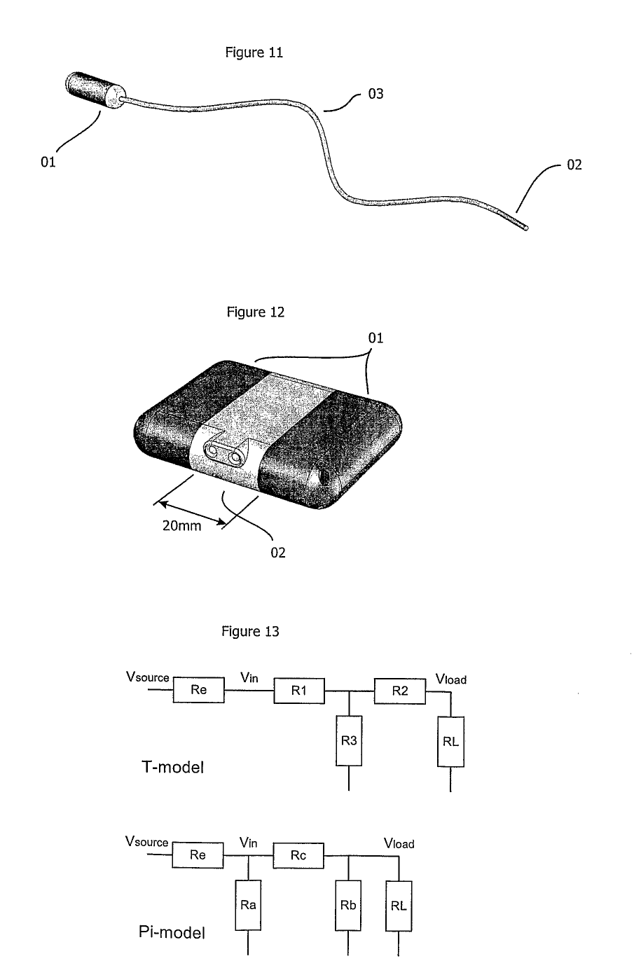

FIG. 11 shows a micro neurostimulator implant with combined receiving and stimulating electrodes

FIG. 12 illustrates an implanted device with integral electrodes in the case.

FIG. 13 shows examples of T and Pi networks used in modeling energy losses in the tissues.

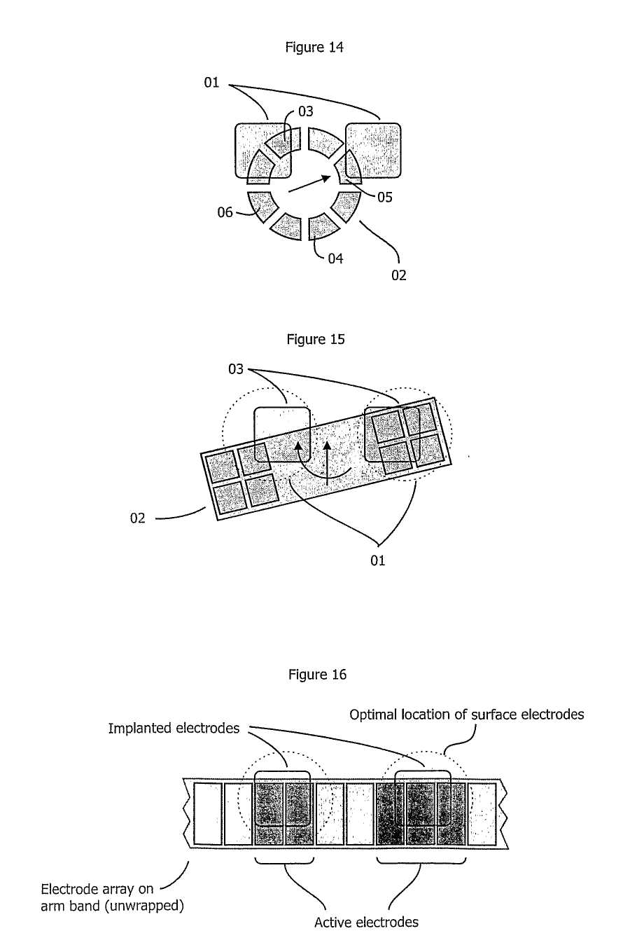

FIG. 14 illustrates a circular array of surface electrodes used to assist in location of the implanted receiving electrodes.

FIG. 15 illustrates a linear array of surface electrodes used to assist in location and then in supplying energy to implanted receiving electrodes.

FIG. 16 illustrates an arm band cut and laid flat with an array of surface electrodes for selective switching of supply current.

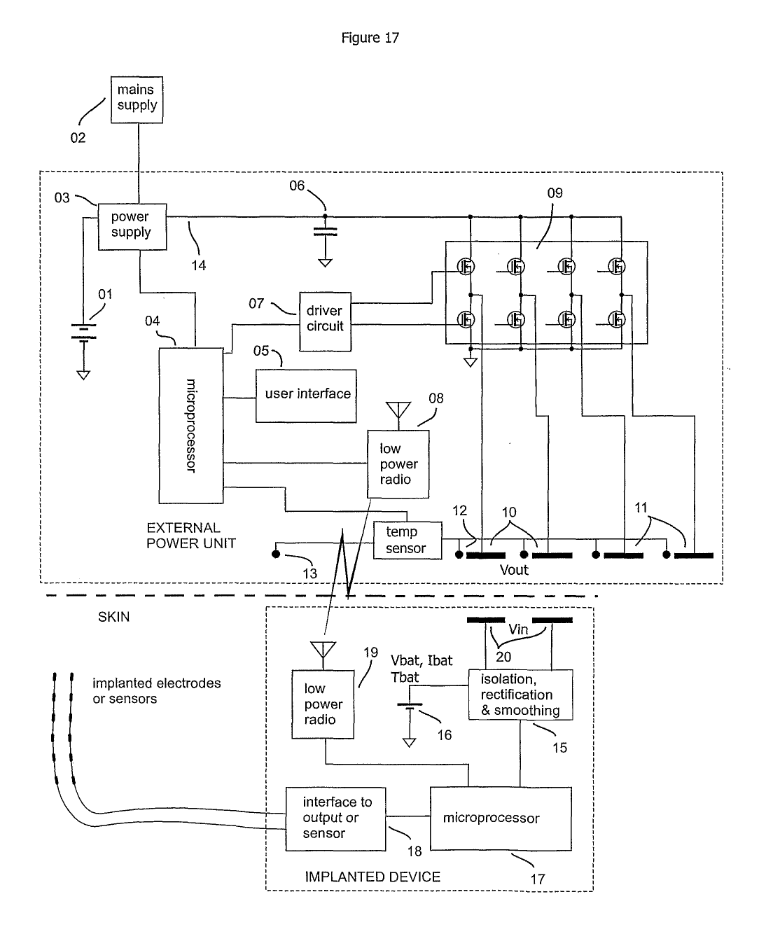

FIG. 17 shows a block diagram of an embodiment of the apparatus according to one aspect of the invention.

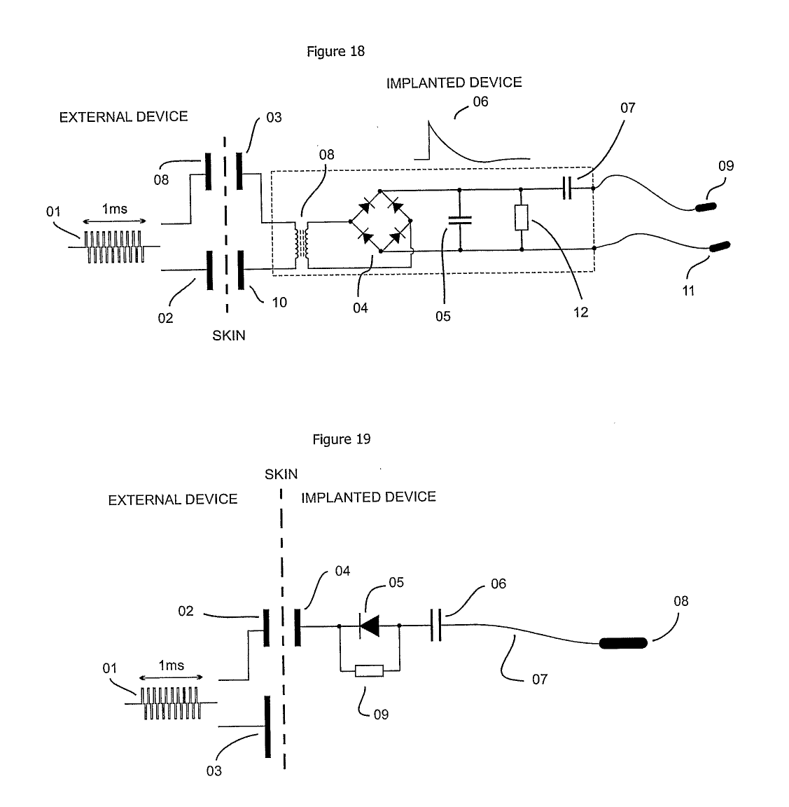

FIG. 18 illustrates a block diagram of a simple single output neurostimulation device.

FIG. 19 illustrates a minimal implanted neurostimulation device.

Referring to FIG. 1, the system comprises an external power unit, 01, that is coupled to surface electrode pads, 02, on the skin. The external power unit provides a stream of pulses of electrical energy, at pulse widths that are shorter than the response time of peripheral nerves at a given voltage.

The implanted components comprise two or more implanted receiving electrodes, 03, which are used to receive the electrical energy transmitted by the external power unit. These are connected by wires to the implanted device, 04, where the electrical energy is conditioned to power the implanted device and any connected devices and/or recharge an internal battery or other electrical storage means such as a capacitor.

The implanted device may either have external contacts as illustrated in FIG. 1, or these may be combined in the case of the unit as illustrated in FIG. 12. It is also possible for one electrode to be positioned directly below the skin and the second electrode provided by the case of the unit.

A key aspect of the invention is that electrical energy is applied directly to the patient's tissues, but for comfort and convenience this energy should preferably be supplied in such a way that the patient is not aware of the electrical impulses. This requires the applied waveform to be either of low amplitude or of pulse duration shorter than the time taken to activate peripheral nerves.

As tissues exhibit a series capacitive reactance, it is preferable that the pulse width of the applied waveform is short enough that the current does not decay appreciably during the pulse. Preferably, pulse widths of under 10 us or more preferably under 5 μs or even more preferably under 2 μs are suitable.

Furthermore, the applied waveform must be such that such that energy dissipated in the tissue does not unduly heat the electrodes, surrounding body tissues or the implanted device. This dictates that the waveform should be either a continuous waveform of amplitude sufficiently low as not to heat the tissues unduly, or a higher power waveform which is interrupted to allow heat to be dissipated. Examples of such waveforms are illustrated in FIGS. 2 to 5.

As illustrated by example in FIG. 2, the waveform may be an interrupted waveform, delivered in single cycles separated by relatively long quiet periods. The pulse may be of square form as shown in FIG. 2, sine wave as shown in FIG. 3, or any other waveform.

FIG. 2 illustrates a waveform with a forward pulse, of pulse width tₚ, and amplitude Vₚ, followed by a balancing negative pulse of identical charge after a period tₛ, referred to as the inter-pulse spacing. The width of the pulse tₚ, is selected as function of Vₚ so that the pulse will not activate peripheral nerves as described below. In a typical application, an automatic control may vary Vₚ in order to deliver a fixed voltage to the implant as desired in the application. The value of the inter-pulse spacing tₛ is typically less than a microsecond for reasons disclosed below. The cycle repeat time, t_{c}, is long relative to the pulse width in a typical application, selected so that undue tissue heating does not occur. The values of t_{c} and tₚ may be varied by an automatic control to keep tissue heating within a limit, typically 2 or 3 degrees centigrade or thereabouts above the surrounding skin temperature.

It is also possible for the waveform to have no return pulse, termed a monophasic signal. A balancing negative pulse is however desirable to eliminate bulk migration of charged particles such as ions to one or other electrode, which may cause skin rashes. Although shown as a pulse of identical shape, the balancing pulse may also be a very low amplitude pulse of long duration, if necessary occupying the entire period when the signal might otherwise be at zero volts. This configuration is however not the preferred one, since it is difficult to design the implant circuitry to extract energy from this type of return pulse.

FIG. 4 illustrates another type of interrupted waveform, whereby bursts of pulses are delivered separated by quiet periods, the quiet period provided for example to allow heat to be dissipated in the tissues between bursts so that the waveform does not cause undue heating. In this case, the time base in FIG. 4 is significantly longer than that in FIGS. 2 and 3 such that the number of single cycles delivered per second would be broadly equivalent in each case.

According to this invention, it is also possible for the waveform to be continuous waveform, which may be a square wave, sine wave or any other waveform. In the case of a continuous waveform, the amplitude of the signal is normally lower than that of an interrupted waveform because of tissue heating limitations. As an approximation, tissues can be considered to be largely resistive at the frequencies concerned, so power is approximately proportional to duty cycle and also proportional to the square of the applied voltage. Consequently, assuming tissue impedance is the same in both cases a 4% duty cycle, 100 V peak, waveform illustrated in FIG. 2 is broadly equivalent in power to the 50% duty cycle, 28 V waveform illustrated in FIG. 5. Of course, the waveform need not be of the square wave form illustrated in FIG. 5, any other form of repetitive waveform is applicable, such as the sinusoidal waveform illustrated in FIG. 6.

One feature of the continuous waveforms illustrated in FIGS. 5 and 6 is that voltage at the implanted receiving electrodes is generally a few volts or less and therefore a transformer or other step-up circuit is required to transform the received signal into a voltage that is readily useable in the implanted device. At high frequencies, such a transformer would however be quite small and would have a relatively high efficiency. It is also desirable to incorporate a transformer in certain applications, such as neurostimulators since this provides isolation between the implanted receiving electrodes and the stimulating electrodes which allows both to be operated simultaneously without coupling between them. Therefore, the continuous waveforms in FIGS. 5 and 6 may be preferred in applications where an isolating transformer is a requirement of the application as this simplifies the electronics of the external power unit.

In each case in FIGS. 2-6 inclusive, the axes have been labeled to indicate typical pulse widths and voltages, but these should not be construed as limiting. For instance, it is relatively common for the peak to peak applied voltage to be as high as 500V.

As previously mentioned, a key aspect of the invention is that electrical energy is applied directly to the patient's tissues, but for comfort and convenience this energy must be supplied in such a way that the patient is not aware of the electrical impulses.

The strength-duration curves observed by Li et al 1976, describe the amplitude required, for any given duration of a single pulse applied to a dissected nerve, to produce an action potential recorded from that nerve. Their observations, and the generally accepted view today, are that the strength-duration curve indicates that the stimulus current and duration can be mutually traded off over a certain range.

Table 1 shows threshold of sensation as a function of pulse width and pulse amplitude derived using surface electrodes. Two self-adhesive electrodes each 50 mm square (Axelgaard PALS platinum) with centers 100 mm apart were placed on the lateral aspect of the abdomen level with T11. This is representative of a typical implant location for the implanted pulse generator in a spinal cord stimulation device.

At each pulse width the amplitude in milliamps (zero to peak) of a pulse that just causes sensation in one subject (the author, a 43 year old healthy male) was recorded. The voltage waveform used was a symmetrical biphasic waveform of similar form to that illustrated in FIG. 2 but with zero inter-pulse space. The pulse width, tₚ, was varied as specified in Table 1. In practice, the pulse current falls away due to the series capacitance of the tissues and also the method of delivery of charge used in the experimental apparatus, which was to charge up two capacitors, one to the deliver the forward pulse and one to deliver the reverse pulse, and discharge these into the tissues. Consequently, the peak current at the start of the pulse was recorded. This decays by approximately 25% at the end of a 2 μS pulse, and 80% at the end of a 20 μS pulse.

The experiment was repeated at three cycle repeat periods, t_{c}: 0.4 ms (equal to a cycle frequency of 2500 Hz); 1 ms (1000 Hz) and 10 ms (100 Hz).

TABLE 1
Peak pulse current (mA) required to produce the onset of sensation at
various pulse widths and cycle repeat periods employing a symmetrical
biphasic waveform with zero interpulse spacing

| Pulse | Cycle repeat period | | |
|---|---|---|---|
| Width, μs | 0.4 ms | 1 ms | 10 ms |
| 2 | 1180 | 1100 | 1150 |
| 3 | 728 | 656 | 624 |
| 4 | 520 | 456 | 392 |
| 5 | 405 | 312 | 276 |
| 6 | 288 | 236 | 204 |
| 8 | 196 | 158 | 142 |
| 10 | 118 | 108 | 96 |
| 20 | 58 | 44 | 44 |

The results in Table 1 are illustrated graphically in FIG. 7. By inspection of the graph and the table above, it is apparent that the current required to produce sensation appears to vary little with cycle repeat frequency. The average current flowing (adding the modulus of the positive and negative cycles) is proportional to cycle repeat frequency, but this seems to have little effect on the onset of sensation. It seems that the onset of sensation is a function mainly of the amplitude of individual pulses. It is largely independent of the number of times that pulse is repeated for repetition times between the 0.4 and 10 ms values shown, but there is a small variation with the shorter cycle repeat times, requiring a higher current to cause the onset of sensation. This may be related to the physiological limits which nerves can follow, which is generally accepted to be in the region of 800-1200 Hz. For the case of the 0.4 ms cycle repeat time, the stimulation is delivered at 2,500 kHz. This is however a useful result for this invention, since it means that the deliverable current at short cycle repeat times is higher than simple proportionality with frequency would suggest.

The graphs are approximately straight lines when plotted on a log-log scale, which gives rise to the approximation Iₛ=ktₚ^{m}, where Iₛ is the peak pulse intensity in milliamps, tₚ is the pulse width and k and m are constants.

For the case of the case of a 0.4 ms cycle repeat period k=3000 and m=−1.3. In practice, the external power unit is designed to deliver a maximum peak pulse intensity in the region of half this approximation, i.e. Iₛ=1500 tₚ^{-1.3}. This ensures that it is possible to control the charge delivered to the implant device by changing repetition frequency, without stimulating peripheral nerves and without causing a distracting tingling sensation for the patient.

In the case above, a biphasic waveform with zero inter-pulse spacing was used. The choice of a small or zero inter-pulse spacing is important since the presence of the reverse pulse tends to suppress activation of the nerve. This is illustrated in table 2, which compares two square waveforms, both of 0.4 ms cycle repeat period. The biphasic case has a reverse pulse with zero inter-pulse spacing. The monophasic case omits the return pulse. Electrode placement was as Table 1 above. N/S means no sensation within the 250 V limit of the output voltage of the experimental device. By inspection of the table, it is apparent that at pulse widths below 5 μs, the biphasic balanced waveform can deliver more than three times the current before the onset of sensation.

TABLE 2
comparison of biphasic and monophasic 4 μs
waveforms or 0.4 ms cycle repeat period.

| | Current (mA zero to | | Reduction |
|---|---|---|---|
| Pulse | peak) at threshold of | | in current |
| Width, | sensation | | at onset of |
| μs | Biphasic | Monophasic | sensation |
| 0.5 | N/S | 1420 | — |
| 1 | N/S | 784 | — |
| 1.5 | N/S | 480 | — |
| 2 | 1180 | 354 | 70% |
| 3 | 728 | 222 | 70% |
| 4 | 520 | 160 | 69% |
| 5 | 405 | 132 | 67% |
| 6 | 288 | 126 | 56% |
| 8 | 196 | 92 | 53% |
| 10 | 118 | 82 | 31% |
| 20 | 58 | 43 | 26% |

A further aspect of this invention is that as the inter-pulse spacing is increased, the suppressing effect of the reverse pulse reduces and it greatest at zero inter-pulse spacing. There is a rapid reduction in the current at which first sensation is felt as inter pulse spacing is increased from zero to approximately 50 μs, after which sensation is virtually independent of inter pulse spacing.

These results demonstrate that it is possible to deliver high average currents at high voltages to the tissues independently of sensation. This allows electrical energy to be transferred to the tissues limited only by heating effects in the tissues and electrodes.

### Tissue Impedance Effects

FIG. 8 shows a graph of impedance of the tissues measured over the median nerve using stainless steel mesh electrodes of size 50×50 mm with a variable frequency sine-wave input. As a useful approximation, the impedance looks like a series R-C combination, as shown in FIG. 9. This neglects the roll-off at high frequencies which is due to the capacitance of the lead and electrodes used in the experimental apparatus. A parallel resistor of high value could be added to the equivalent circuit to simulate the DC body DC resistance, although this is several tens of thousand of ohms as a minimum and can therefore also be neglected for the purposes of this invention. By inspection of the graph, the tissues can be considered to be primarily resistive between 25 kHz and 10 MHz.

The typical value of bulk tissue resistance with pulses of 0.5 to 4 μs is 150Ω, measured with stainless steel mesh conductor electrodes at 200 mm centre distance. This varies surprisingly little with electrode placement, suggesting that the bulk of the tissues beneath the pads are responsible for conduction, rather than conduction in the skin or subcutaneous fatty tissues. Living human tissue and pig meat used for some of the experiments described below have similar characteristics. The measurement is readily taken by applying a 0.5 μs 200 V square wave pulse and measuring the peak pulse current at the start of a pulse.

Energy Transfer from Surface to Implanted Electrodes.

Tests on the implanted electrodes were performed on fresh cuts of shoulder of pig, mass 7 kg, and leg, mass 6 kg. The shoulder represents a cut with a higher fat content than the leg. As a first step, the bulk tissue impedance was measured and confirmed to be approximately 150Ω, in both cases the same as typical for living human tissue.

The relative resitivity of the tissues, samples of skin, fat and lean meat are 1580 Ω-cm for the skin, 435 Ω-cm for fat and 180 Ω-cm for red meat. This suggests that the current flows not only through the skin or subcutaneous fat as might be assumed, but also through the lower resistivity flesh below. The author has verified this experimentally by measuring bulk impedance between two electrodes and then forming a 10 mm wide gulley around one electrode by removing skin and subcutaneous fat, so that the current must flow directly through the tissues below. The effect on bulk tissue impedance with a 8 mm skin and fat layer was to increase the impedance from 140Ω to 150Ω, measured with 50 mm square electrodes 200 mm apart using a 0.5 μs biphasic pulse with a cycle repeat period of 100 μs.

The distribution of current in the tissues is such that using surface electrodes as above with implanted electrodes of 42 mm square directly below the surface electrodes, approximately 65% of the voltage applied to the surface electrodes appears across the implanted electrodes with an implantation depth of 5 mm and a high impedance measuring device. In deeper tissues, the voltage falls away, In the shoulder of pig which had an intact spinal cord, approximately 7% of the applied voltage was detected with electrodes over the spinal cord approximately 150 mm below the surface of the skin. These are open loop measurements of voltage, and the voltage is reduced once a load is attached to the electrodes; nevertheless this voltage would be sufficient to power sensors or other low power devices buried in deep tissues. Such devices work better if the receiving electrodes are separated. Such devices may incorporate two receiving electrodes, one the case of the implant and the other on the end of a wire which may either double as a connection to a sensor or a simulating electrode, or may operate as a power receiving electrode just below the surface of the skin.

This is illustrated in FIG. 11, which shows a micro neurostimulator implant with combined receiving and stimulating electrodes. Referring to FIG. 11, the device is small (the length of the body of the implant may be typically 10 mm) to allow implantation for instance in the head or neck for deep brain stimulation. In normal operation, the implant case, 01 forms a grounding electrode and the other electrode, 02, which is the active stimulating electrode, is located on the end of the insulated wire, 03 and implanted as required.

To charge an internal battery in the implanted device, one surface electrode is placed over the skin near the site of implant of the case, 01, and the other surface charging electrode is placed elsewhere on the body so that the potential difference between the ends of the implant is maximized. The relatively large physical separation of the case, 01, and electrode, 02, allows a significant potential difference to be produced between the two electrodes. Of course, the waveforms described herein are such that the charging waveform is of sufficiently short pulse width that it does not stimulate the tissues, whereas the stimulating waveform produced by the implant would be of much longer pulse widths to produce the desired therapeutic result by activating nervous tissues. A variation on the implant in FIG. 11 is to put an additional contact along the wire, 03, at the end near the stimulating electrode to provide a dedicated connection for the charging circuit. The advantage of this approach is that it is possible to provide a larger contact area for the charging electrode, which improves efficiency, while the stimulating electrode can be kept very small so that the stimulus current can be delivered to a precise location. It is also possible to charge the device and provide stimulus independently using this method.

The effect of having one surface and one deeply implanted receiving electrode is that efficiency is reduced by a factor of approximately one half compared to a configuration where both receiving electrodes are just under the surface of the skin.

Another aspect of the invention described below relates to higher power implant devices such as spinal cord stimulators. To maximize the efficiency of energy transfer between an external power unit and the implant device, it is preferable that the surface electrodes and implanted receiving electrodes are in close proximity. To allow the implant device to be sited in deeper tissues, so that it is invisible under the skin, it may be preferable to have electrodes connected to the implant via wires are shown in FIG. 1. The receiving electrodes can be positioned just under the skin and may be constructed of a flexible material such as a mesh or other thin biocompatible conductive material such as a fine layer of platinum deposited on a flexible substrate which flexes with the skin. An example of such an electrode is illustrated in FIG. 10. The electrodes may be sited in an area that is convenient for attachment of a charging unit but would not be cosmetically suitable for implantation of the device itself, such as the upper arm or leg, so that the external power unit can be attached by means of a leg or arm strap which has surface electrodes integrated into the strap.

Referring to FIG. 10, the electrode consists of a conducting surface, 01, connected to a wire, 04 that is connected to the implanted device. The conducting surface overlies an insulating substrate, 03. Preferably the conducting surface and insulating substrate are flexible so that the electrode can move with the skin and tissues. One or more holes, 02, may be provided in the electrode to permit it to be sutured in place. The electrode is implanted under the skin with the conducting face proximate to the skin.

The reason for incorporating the insulating layer is that the efficiency of energy transfer from surface to receiving electrodes is improved by insulating the bottom surface, but this is only applicable for electrodes that are implanted at a depth under the skin less than about half their smallest dimension across the conducting face.

For implantation below this depth, the additional area of the electrode provided by having both sides conducting is advantageous. Additionally, electrodes formed from a conducting woven material, such as a wire mesh, are advantageous because of their large effective surface area, flexibility and tendency to bind with the tissues and therefore not migrate after implantation.

If it is possible to implant the device proximate to the surface of the skin, then integrated electrodes may be preferred even for devices with relatively high output. In the case of an implant device with integrated electrodes, efficiency is maximized when the electrode area is relatively large. This gives rise to configurations such as illustrated in FIG. 12 wherein the receiving electrodes, 01, are formed from the ends of the implant casing with an insulating material, 02, separating them and providing exits for connectors etc. The width of the insulating section need not be greater than 20 mm or thereabouts, as a large electrode area is more important than separation between the electrodes for efficient transfer of energy from the tissues.

### Model of Tissue Losses

For the purposes of understanding losses in the system, it is useful to make the assumption that the tissues can be represented using resistive elements only (i.e. no reactive elements). This assumption is an approximation because it neglects the capacitive coupling (50 nF series element) as represented in FIG. 9 but is probably adequate for predicting energy transfer efficiency. The losses in the reactive element(s) will be small at the pulse widths proposed.

The connection between the skin surface and the surface of the buried electrodes can be modeled as a T (Y) network or as a Pi (delta) network as illustrated in FIG. 13. The two networks are topologically different but are electrically identical when viewed from their terminals.

We tried a number of implant configurations and produced solutions for the resistor network:
Source electrodes 15×22 mm and target electrodes 10×20 mm on 50 mm centers, receiving electrodes just below the skin, electrode backs insulated from flesh below.
Source electrodes 50×50 mm and target electrodes 10×20 mm on 50 mm centers, receiving electrodes just below the skin, electrode backs insulated from flesh below.
Source electrodes 50×50 mm and target electrodes 42×42 mm on 72 mm centers (30 mm edge to edge), receiving electrodes below the skin, electrode backs conducting and connected to electrode tops.
Same as above but spacing of sending electrodes increased to 102 mm centers (60 mm edge to edge).

In each case a number of different load impedances were applied to the implanted electrodes.

Solving the resistor values for a range of implant types yields useful data about power losses. Specifically, they indicate that most power is dissipated on the left hand side of the network, approximately equivalently in the shunt and series elements. This is because although the skin and fat have the highest resistivity they also have the largest voltage gradients across them.

Both models have in common that the majority of power is dissipated on the left hand side of the network, primarily because the voltage decreases rapidly as the signal penetrates the tissues. For example, surface electrode contact is important. In the experimental apparatus, the surface electrodes had a resistance of 13Ω and accounted for losses amounting in the range 10% to 14% of the input power.

The overall transfer efficiency varies with many factors such as implanted electrode size, surface electrode size and location, and most importantly the depth of the implanted electrodes.

Maximum transfer efficiency into the load occurs when the load impedance is equal to the source impedance looking back from the right hand side of the network. The source impedance is made up of the impedance of the external stimulator output, the electrodes and the various series and shunt elements in the tissues.

Also important is load impedance, which varies with the electrode configuration and tissues but is typically optimum in the region of 50Ω to 150Ω. Transfer efficiency in an optimal system comfortably exceeds 10%.

### Methods of Locating Implanted Electrodes

Transfer efficiency is not particularly sensitive to surface electrode location, provided that the surface electrodes are generally aligned with the implanted electrodes and spaced slightly further apart, or over the top of, the implanted electrodes. Methods of locating and aligning the surface and implanted electrodes are described below.

FIG. 14 illustrates a circular array of surface electrodes, 02, on an external device, used to assist in location of the electrical center of the implanted receiving electrodes, 01. In this example, the implanted electrodes are within 2-3 cm of the surface of the skin and separated typically by 2-3 cm between their adjacent edges. Detection of the location and orientation of the implanted device can be accomplished by making contacts 03 and 04 common, and selectively applying a signal to contact 05 and then contact 06. This is repeated around the array in a circular fashion to yield the direction and orientation of the implanted electrodes with respect to the surface array. The voltage at the implant is sensed for each signal combination and information transmitted from the implant to the external device.

Alternately, the external device can operate without communication with the implanted device by measuring changes in impedance of combination of tissues and device at the array. A display on the external device directs the user to move the device until it is over the electrical centre of the electrodes. The display would also indicate the orientation of the implanted electrodes. The contacts on the external device would be metallic contacts and a gel may be used if required to improve electrical contact with the skin.

FIG. 15 illustrates another linear array of surface electrodes used to assist in location and in supplying energy to implanted receiving electrodes. In this example, the optimal positioning of the surface electrodes relative to the implanted electrodes is indicated by the dotted line, 01. In this case the implanted electrodes are under a relatively flat area of the skin, which gives rise to an optimal position for the surface electrodes either directly over or separated slightly more than the implanted electrodes, 03.

By selectively stimulating the electrode arrays, 02, which are part of an external power unit, it is possible to direct the user by means of a display to place the external power unit over the implanted electrodes and in line with the implant. Once the external power unit is in position, the user secures it in place and electrodes in the array are used to transmit energy to the implant.

FIG. 16 illustrates part of an arm, leg, or neck band containing the stimulating electrodes, cut and laid flat for clarity. In this example, the band is simply positioned on the arm, neck or leg at the correct position and the device tests combinations of contacts in turn until it finds an optimum combination to deliver energy to the implant at maximum efficiency. In this illustration, the active electrodes are indicated by the darker grey shading. The advantage of the arm, leg or neck band is that it is simple to use, requiring no manual adjustment of position, easy to fit and the efficiency of transmission is high because the implanted electrodes may be separated on each side of the arm, leg or neck.

It will be appreciated by those skilled in the art that the electrode arrays described above may be of many other configurations and the detecting signals may be applied either systematically as described or in a random or pseudo-random sequence. Indeed it may be desirable to individually control amplitude and phase of the driving signal for each electrode to maximize energy transmission to the implant and that the coefficients for these driving signals may be derived by using known signal processing techniques.

### Typical Implant System

FIG. 17 shows a block diagram of a typical apparatus according to the invention. It is desirable that the patient is able to move around while charging the implanted device, so that external power unit is provided with rechargeable batteries, 01, which are charged from a mains supply 02, when the external power unit is not being used by the patient.

The external power unit in this example is designed to produce a biphasic square wave output of variable amplitude, pulse width and cycle repetition rate of the form illustrated in FIG. 2.

Referring back to FIG. 17, the batteries, 01, feed a power supply, 03, which drives a DC bus, 14, at an output voltage typically in the range 25V to 250 V. The power supply is controlled by the microprocessor, 04, to provide the required DC voltage that defines the amplitude of the pulse output. The power supply is designed such that it can only generate a continuous output power that is equivalent to the average maximum output power of the unit. This has significant safety advantages since it means that the output voltage will be quickly pulled down to a safe value in the event of any fault condition in the external device which has the effect of connecting the power supply dc bus to the patient.

The DC bus charges a capacitor, 06, sized so the maximum stored energy and thereby deliverable to the patient in the event of a fault condition cannot exceed 300 mJ. This is the maximum recommended by IEC 60601-2-10, “Particular requirements for the safety of nerve and muscle stimulators”.

The capacitor supplies the pulse current for the output. Because safety considerations dictate that the capacitor and power supply cannot store enough charge to sustain a dangerous voltage on the output beyond the energy limitation specified above, there will be some decay in the DC bus voltage during the forward pulse, and the voltage will not recover completely in time for the reverse pulse. With the large mark-space ratio of atypical waveform as illustrated in FIG. 2, the voltage must however recover fully by the end of the cycle repeat time.

As previously stated, it is desirable that there is no net DC current flow between the surface electrodes to avoid migration of ions under the skin. The preferred means of ensuring that the net DC current is zero is to alternate the polarity of the leading pulse from positive to negative. Alternately, the reverse pulse can be extended slightly so that the net charge is zero. Yet another technique is to provide a capacitor for each half bridge, fed through diodes from the DC bus.

The microprocessor, 07, uses internal counter-timer units to synthesize logic level signals that represent the desired pulse width and cycle repeat period.

The output stage consists of a driver circuit, 07, and an output stage, 09, which in the example illustrated is a bridge configuration consisting of four half bridges for each of four surface electrodes.

The driver circuit has two primary functions. It transforms the local level signals from the microprocessor into drive signals for the transistors in the output stage. It also provides a safety limit in hardware ensuring that no pulse can exceed a maximum time, typically 20 μs, even in the event of microprocessor failure. This second feature, combined with the energy storage limit on the capacitor, 06, further ensures that the device cannot deliver a dangerous shock to the patient, even under fault conditions. In a typical embodiment there would also be an additional safety circuit, not shown, that would act as an independent hardware limit in the event that the DC bus exceeds a safe maximum value.

The surface electrode array is an array of two or more surface electrodes, 10 and 11, which may be part of the external power unit, or connected by a wire or wires or may form part of a garment such as an arm band.

As previously indicated, the application may require that there are a plurality of surface electrodes, for instance in order to steer the current path in the tissues so that the energy is automatically aligned with the implanted receiving electrodes, 20, such as is illustrated in FIG. 16.

Referring back to FIG. 17, in this instance, the output circuit, 09, is shown with four surface electrodes but it will be appreciated that if additional surface electrodes are required, they would require additional half bridges so that they can be independently controlled by the microprocessor. In the example illustrated, surface electrodes in the array labeled 10, positioned over the implanted receiving electrodes may be active and those labeled 11 may be inactive.

Proximate to the surface electrodes 10 and 11, but preferably in contact with the surface of the skin, there are one or more temperature sensors, 12, and one or more additional temperature sensors, 13, in a position away from the electrodes, so that the microprocessor can compare the temperature of the skin at the active surface electrodes with the skin temperature away from these electrodes. The difference in these values is used to calculate the degree of heating of the tissues caused by current supplied by the External Power Unit. The microprocessor modifies either the pulse width or cycle repeat time to ensure that the degree of heating is controlled within a safe limit, while attempting to supply energy to the implanted device at the desired rate. In the case of a battery charging application, during the fast charge phase the requirement is generally to provide maximum energy transfer within the heating limit.

The implanted device has two or more receiving electrodes, 20, which are as previously indicated either part of the case of the device or connected by leads. The received signal is rectified and may be smoothed and optionally may be coupled via an isolating transformer, 15 and then may be used to charge an internal battery, 16, in the implant or to directly power the device. One advantage of this invention over induction coil devices is that it is relatively easy to generate the voltages required to control a microprocessor or other electronic device using only passive components in the rectification and smoothing stage, and therefore recovery from a zero volt battery condition is possible, whereas in induction loop devices it may not be possible.

The microprocessor, 17, in the implanted device has two primary functions. Firstly, it communicates with the external power unit via radio, 19, or some other means. Secondly, it provides the interface to the implant output and/or sensing devices. During battery charging, one function of the communication system is to feed back the received voltage on the implanted receiving electrodes, Vin, to the microprocessor in the external power unit, together with other information such as battery state, including the battery voltage Vbat and battery current Ibat, and battery temperature Tbat. With this information, the external microprocessor, 04, is able to adjust the amplitude of the applied voltage so that the correct voltage for battery charging and/or operation of the implant electronics is received at the implant.

The external microprocessor, 04, contains three primary control loops. The first implements control of pulse amplitude based on feedback of received voltage, and adjusts either the cycle repeat period or the pulse width to keep power delivered within appropriate limits. The second controls the power delivered to keep it at the desired level for battery charging without undue heating, based on feedback from the temperature sensors. The power delivered is controlled by either changing pulse repeat period or pulse width or both. The third supervisory loop monitors the pulse width and amplitude to ensure that these are maintained below the level that might cause activation of peripheral nerves, so that the electrical current cannot be felt by the patient.

The external microprocessor also contains a number of state machines that control overall function of the system. An example is the battery charging state machine. The purpose of this is to ensure that the implanted battery is fully charged. In order to properly charge the internal battery, it is first necessary to determine whether the battery can be fast charged or should be trickle charged. Trickle charging is important either when the battery is very low, near 0 V, or when the battery is nearly fully charged. With feedback of battery voltage Vbat, battery current, Ibat, battery temperate, Tbat, the external microprocessor is able to determine how to control Vin to deliver the correct battery charging current. This allows the rectification and smoothing stage, 15, in the internal device to be very simple, at its simplest consisting of a bridge rectifier and a smoothing capacitor.

It has previously been mentioned that in order to maximize power transfer to the implanted device, the impedance of the input to the electrodes should ideally be in the range 50-150 ohm and may need to be controlled in order to maximize power transfer. Clearly a variable impedance isolation, rectification and smoothing stage is desirable, and there are a number of known techniques for achieving this, however in the simplest case the impedance can be controlled by the external microprocessor by controlling the difference between the applied voltage on the electrodes and the battery voltage, given a certain internal resistance of the battery. The technique, while having certain limitations, gives rise to a very simple implant device and battery charging circuit since the majority of the complex electronics is sited in the external unit. Simplicity of the implant device is highly desirable for longevity.

### Minimal Neurostimulation System

FIG. 18 illustrates a very simple single output neurostimulator based on this invention.

The external device produces a burst waveform, 01, consisting of a burst of repeated short pulses of 1 μs duration forward pulse, a 1 μs interpulse spacing, a 1 μs reverse pulse and a further a 1 μs space before the next forward pulse, typically lasting one millisecond and repeated at 100 Hz. A typical location for the external stimulator would be the upper arm, as it is convenient to build an external device which is part of an armband that is worn whenever stimulation is required.

The signal is delivered in bursts which are repeated at 100 Hz, and applied to the skin via two or more surface electrodes, 02 and 08. Across the upper arm, such a burst waveform may be delivered at 230 V, with pulse current in the region of 1.5 A or more, with no sensation of tingling or heating in the arm.

The implanted electrodes, 03 and 10, are positioned on either side of the arm under the skin. They are formed of a flexible material so that they are not visible and flex with the skin. The applied signal is received by these electrodes and fed through an isolation transformer, 08, into a bridge rectifier, 04 and smoothing capacitor, 05. The capacitor is charged by the rectified train of signals from the input waveform, but is simultaneously discharged into the tissues. This gives rise to a signal with a relatively rapid increasing phase and an exponential decay, 06.

By omitting the series capacitor, 07, and resistor, 12, this signal may be applied directly to the stimulating electrodes, 09 and 11, which are implanted in target nervous tissue. In the example given, the signal will activate nervous tissue at an amplitude of typically 25 V at the implanted electrodes. This amplitude on the output is readily achievable assuming a 10% transmission efficiency from surface to implanted electrodes.

Alternately, if a stimulus signal with no net DC component is required for instance to minimize ionic transport an optional series capacitor, 07, may be added to the output. In this case, a resistor, 12, is provided in parallel with the bridge rectifier. This is a high value resistor in comparison with the resistance of the tissues, selected to provided a return path for the charge in the series capacitor and smoothing capacitor if fitted, in the period between each burst waveform, 01. An incidental effect of the series capacitor will be to reduce the amplitude and width of the stimulus pulse.

The isolating transformer is optional, but may be provided to prevent coupling between the receiving and stimulating electrodes in the implant, although it should be noted that these can never be completely decoupled as they are always connected by the tissues. Clearly, this isolation could be at the output rather than the input to the stimulator. Input isolation is preferred because the transformer is operating at higher frequency and is therefore smaller.

Such a minimal neurostimulation system has the advantage that the implanted electronics are extremely simple and therefore could reasonably be expected to have a service life of 20 years or more. Furthermore, as the circuit is very compact, typically 2 cm³, it can be combined if required within one of the implanted receiving electrodes.

It can be seen that this minimal system suffers from a number of limitations; in particular there is no control of input impedance, no feedback of received voltage at the implanted receiving electrodes and only one output. Clearly, the provision of a more sophisticated rectification and smoothing stage, a microprocessor and low power radio would allow this additional functionality to be incorporated using known techniques with only a minimal increase in the size of the implanted device. It would then be possible to incorporate a small battery and allow the implanted device to operate when the external power unit is not attached. These types of configurations have been discussed elsewhere in this document.

Further simplification of the system described above is possible, by for example omission of the isolating transformer, 08 and the smoothing capacitor, 05. Such a system is compact, efficient and functional. Additionally, it is possible to omit the series capacitor, 07 and the resistor 12 and still produce a functional system, although these omissions result in a system that produces a net DC output at the stimulating electrodes which is not a preferred configuration.

Ultimately, a working system may be reduced to that illustrated in FIG. 19. Referring to the figure, a burst waveform, 01, is generated by the external device and applied to two electrodes on the skin, a supply electrode, 02 and a return electrode, 03. These are of substantially greater surface area than the smaller implanted stimulating electrode, 08. The implanted device is very simple, consisting of single implanted receiving electrode, 04, a rectifying diode, 05, an optional series capacitor, 06, coupling the output from the diode to the stimulating electrode, and a resistor, 09, sized as before to provide a return path for the charge in the series capacitor during the period between each input pulse.

An insulated lead, 07, allows the stimulating electrode to be positioned as required in the tissues, which lead may be flexible or rigid as required by the application.

This arrangement provides a single point of stimulation in the tissues. Because of the very small size of the implanted electronics, only a minimally invasive procedure is required to implant the device. For convenience, the external device may be constructed in a single unit that is attached to the skin via self adhesive electrodes over the implanted receiving electrode.

In some applications, it may be desirable to have one or more electrodes on the insulating lead, 07. These are typically arranged as a series of cylindrical stimulating electrode contacts in a line along the insulating lead. This lead is typically 1 to 2 mm diameter, each contact typically 1 to 5 mm in length and separated by 0.5 to 5 mm. Each contact is connected via a wire in the insulated lead, to a respective implanted receiving electrode, series diode and capacitor of the configuration illustrated in FIG. 19. The receiving electrodes (one for each contact) are arranged just under the skin in a linear array or other convenient arrangement. A corresponding arrangement of supply electrodes is placed on the skin over the implanted receiving electrodes.

In this example, it may desirable to control the amplitude of the stimulating current or voltage applied to each stimulating electrode contact, as this allows the effective position of the stimulating signal to be varied along the insulating lead. This may be important when attempting to focus stimulation on a small area of excitable tissue. By modulating the amplitude of the burst waveform generated by the external device applied to each of the supply electrodes, it is possible to control both the amplitude and the effective position of the stimulating current developed at each of the series of cylindrical stimulating electrode contacts.

What is claimed is:

1. An implanted system comprising:
a power device external to a human or animal body, the power device having power electrodes in contact with the body so as to make direct electrical connection to the tissues of the body, the power device being arranged to apply electrical energy to the body via the power electrodes; and
an implanted device within the body, the implanted device having implanted electrodes arranged to receive said electrical energy via the body, thereby to provide power for said implanted device,
wherein the power device is arranged to apply an electric potential between the power electrodes, the potential being in the form of a repetitive waveform, the electric potential being such as to generate electric current in the body and thereby to transfer said electrical energy to said implanted electrodes.

2. A system according to claim 1, wherein the repetitive waveform is a pulsed waveform.

3. A system according to claim 2, wherein the pulses of the pulsed waveform have a duration not greater than 8 μs.

4. A system according to claim 2, wherein the polarity of the pulsed waveform alternate between positive and negative pulses.

5. A system according to any one of claims 2, wherein the spacing of the pulsed waveform has an interpulse period not greater than 20 μs.

6. A system according to claim 1, having a temperature sensor for detecting the temperature of the body adjacent the power electrodes, and means for controlling the electric energy applied to the body in dependence on said temperature.

7. A system according to claim 1, wherein the implanted device has means for detecting the electrical power received by the implanted electrodes, means for transmitting to the power device information relating to said received power, and the power device has means for controlling the electrical energy applied to the body in response to said information.

8. A system according to claim 1, wherein the power electrodes are on an external surface of the body.

9. A method of providing power to an implanted device implanted within a human or animal body, the implanted device having implanted electrodes; the method comprising
locating power electrodes of a power device in contact with the body so as to enable direct electrical connection to tissues of the body, the power device being external to the body;
applying electrical energy to the body via the power electrodes by applying an electric potential between the power electrode, the electric potential being in the form of a repetitive waveform, thereby generating electric currents in the body;
receiving said electric current at said implanted electrodes, thereby conveys said electric energy to said electrodes, thereby to power said implanted device.

10. A method according to claim 9, wherein the power electrodes are on an external surface of the body.
